(19) United States
(12) Patent Application Publication
(10) Pub. No.: US 2009/0214511 A1
(43) Pub. Date: Aug. 27, 2009

(54) DIGESTIBLE COMPOSITIONS OF INULIN FOR MANAGING BLOOD GLUCOSE LEVELS

(76) Inventors: Trung Hong Tran, San Jose, CA (US); Thanh Hong, Chantilly, VA (US)

Correspondence Address: Trung Hong Tran, 2551 Palmetta Palm Court, San Jose, CA, 95133, US

(21) Appl. No.: 12/390,385

(22) Filed: Feb. 20, 2009

Related U.S. Application Data
(60) Provisional application No. 61/030,228, filed on Feb. 21, 2008.

(51) Int. Cl. A61K 38/47; A61K 31/733; A61P 3/08

(52) U.S. Cl. 424/94.61; 514/54

(57) ABSTRACT

Digestible compositions of inulin for managing blood glucose levels in a patient are disclosed. The compositions can include an inulin, a sucrose and an amylase, wherein the inulin is configured to lower blood glucose in the patient and wherein the sucrose is presented in sufficient amount to at least partially counteract the blood glucose lowering effect of the inulin. Several embodiments disclosed in the present application include a vegetable mixture comprising burdock root, carrots and daikon radish and additional constituents such as dimethyl sulfoxide, lentinan and D-eritadenine. Additionally, methods of preparing a digestible composition of inulin are also disclosed.

### REFERENCE TO RELATED APPLICATIONS

The present application claims the benefit of U.S. Provisional Application Ser. No. 61/030,228 filed Feb. 21, 2008 and titled “Herbal Health Drink Made From Plant Arctium Lappa”, which is incorporated herein by reference in its entirety.

### TECHNICAL FIELD

The present application generally relates to dietary and therapeutic formulations that include soluble fibers comprising inulin. More specifically, the present application relates to compositions comprising digestible forms of inulin to be ingested for managing blood glucose levels.

### BACKGROUND

Diabetes mellitus is a chronic metabolic disorder that is caused by a failure of the body to produce insulin and/or inability of the body to respond adequately to circulating insulin. The unavailability of useable insulin results in elevated blood glucose levels, which are toxic to the body and can lead to severe complications in the future. Type 1 diabetes occurs most often in children or young adults and accounts for 5-10% of the diagnosed diabetes patient population. Type 2 diabetes, or adult-onset diabetes, accounts for 90-95% of diagnosed diabetes cases worldwide. Now, because of the epidemic of obesity and inactivity in children, type 2 diabetes mellitus is occurring at younger and younger ages. There are currently 20.8 million diabetics in the US. The CDC forecasts that this number will grow by 1.5 million each year and, ultimately, doubling by 2020.

Nationally, the number of diabetics is expected to double by 2020, currently 20.8 million people (7% of the population). The reason for this growth is two-fold: first, the general population is aging and people are more likely to contract diabetes as they get older. Second, the diet and lifestyles of many Americans have caused the population to contract diabetes at a younger age. The CDC estimates that there are an additional 1.5 million Americans who will contract diabetics each year.

Type 2 diabetes is characterized by peripheral insulin resistance with an insulin-secretory defect that varies in severity. For type 2 diabetes mellitus to develop, both defects must exist: all overweight individuals have insulin resistance, but only those with an inability to increase beta-cell production of insulin develop diabetes. In the progression from normal glucose tolerance to abnormal glucose tolerance, postprandial glucose levels first increase. Eventually, fasting hyperglycemia develops as inhibition of hepatic gluconeogenesis declines.

About 90% of patients who develop type 2 diabetes mellitus are obese. Because patients with type 2 diabetes mellitus retain the ability to secrete some endogenous insulin, those who are taking insulin generally do not develop DKA if it is stopped. Therefore, they are considered to require insulin but not to depend on insulin. Moreover, patients with type 2 diabetes mellitus often do not need treatment with oral antidiabetic medication or insulin if they lose weight or stop eating.

Maturity-onset diabetes of the young (MODY) is a form of type 2 diabetes mellitus that affects many generations in the same family with an onset in individuals younger than 25 years. Several types exist. Some of the genes responsible can be detected by using commercially available assays.

In 2005, people with diabetes were estimated to account for 7% of the US population, or approximately 20.8 million people. Of these 20.8 million people, 14.6 million have a diagnosis of diabetes, and diabetes is undiagnosed in another 6.2 million. Approximately 10% have type 1 diabetes, and the rest have type 2. Additionally, an estimated 54 million people have pre-diabetes. Pre-diabetes, as defined by the American Diabetes Association, is that state in which blood glucose levels are higher than normal but not high enough to be diagnosed as diabetes.

The morbidity and mortality associated with diabetes are related to the short- and long-term complications. Complications include the following:
Hypoglycemia and hyperglycemia
Increased risk of infections
Microvascular complications (eg, retinopathy, nephropathy)
Neuropathic complications
Macrovascular disease (eg, coronary artery disease, stroke)

Diabetes is the major cause of blindness in adults aged 20-74 years, as well as the leading cause of nontraumatic lower-extremity amputation and end-stage renal disease (ESRD).

### Treatment

Diabetes mellitus type 2 is a chronic, progressive disease that has no established cure, but does have well-established treatments which can delay and sometimes avoid most of the formerly inevitable consequences of the condition. There are two main goals of treatment: (1) reduction of mortality and concomitant morbidity (from assorted diabetic complications); and (2) preservation of quality of life

The first goal can be achieved through close glycemic control (i.e., to near ‘normal’ blood glucose levels); the reduction in severity of diabetic side effects has been very well demonstrated in several large clinical trials and is established beyond controversy. The second goal is often addressed (in developed countries) by support and care from teams of diabetic health workers (usually physician, PA, nurse, dietitian or a certified diabetic educator). Endocrinologists, family practitioners, and general internists are the physician specialties most likely to treat people with diabetes. Knowledgeable patient participation is vital to clinical success, and so patient education is a crucial aspect of this effort.

Type 2 is initially treated by adjustments in diet and exercise, and by weight loss, most especially in obese patients. The amount of weight loss which improves the clinical picture is sometimes modest (2-5 kg or 4.4-11 lb); this is almost certainly due to currently poorly understood aspects of fat tissue activity, for instance chemical signaling (especially in visceral fat tissue in and around abdominal organs). In many cases, such initial efforts can substantially restore insulin sensitivity. In some cases strict diet can adequately control the glycemic levels.

Treatment goals for Type 2 diabetic patients are related to effective control of blood glucose, blood pressure and lipids to minimize the risk of long-term consequences associated with diabetes. They are suggested in clinical practice guidelines released by various national and international diabetes agencies.

The targets are: (1) Hb_{A1c} of 6.0% to 7.0%; (2) Pre-prandial blood glucose: 4.0 to 6.0 mmol/L; (3) 2-hour postprandial blood glucose: 5.0 to 8.0 mmol/L.

### Self Management

Diabetes self-management education is an integral component of medical care. Among adults with diagnosed diabetes, 12% take both insulin and oral medications, 19% take insulin only, 53% take oral medications only, and 15% do not take either insulin or oral medications.

Traditionally, information regarding diabetes would be obtained from a family physician. However, with access to the internet so widely available now, people are able to educate themselves through websites. This information can be beneficial, but care must be taken to ensure the information is medically sound. Several of the external links below provide information about diabetes and its management, including self-management.

### Self Monitoring of Blood Glucose

Self-monitoring of blood glucose may not improve outcomes in some cases, that is among “reasonably well controlled non-insulin treated patients with Type 2 diabetes”. Nevertheless, it is very strongly recommended for patients in whom it can assist in maintaining proper glycemic control as it is the only source of current information on the glycemic state of the body, as changes are rapid and frequent, depending on food, exercise, and medication (dosage and timing with respect to both diet and exercise), and secondarily, on time of day, stress (mental and physical), infection, etc.

### Dietary Management

Modifying the diet to limit and control glucose (or glucose equivalent, eg starch) intake, and in consequence, blood glucose levels, is known to assist type 2 patients, especially early in the course of the disease's progression. Additionally, weight loss is recommended and is often helpful in persons suffering from Type 2 diabetes for the reasons discussed above.

Several dietary modifications using dietary supplements are sometimes recommended to those with Type 2; there are studies suggesting that there is some beneficial effect for some of these. See the discussion below.

### Exercise

In September 2007, a joint randomized controlled trial by the University of Calgary and the University of Ottawa found that “Either aerobic or resistance training alone improves glycemic control in Type 2 diabetes, but the improvements are greatest with combined aerobic and resistance training than either alone.” The combined program reduced the HbA1c by 0.5 percentage point. Other studies have established that the amount of exercise needed is not large or extreme, but must be consistent and continuing. Examples might include a brisk 45 minute walk every other day.

### Pharmacologic Intervention

There are several prescription drugs available for Type 2 diabetics. One of the most widely used drugs is Biguanide metformin, which works primarily by reducing liver release of blood glucose from glycogen stores and secondarily by provoking some increase in cellular uptake of glucose in body tissues. Both historically, and currently, the most commonly used drugs are in the Sulfonylurea group, of which several members (including glibenclamide and gliclazide) are widely used; these increase glucose stimulated insulin secretion by the pancreas and so lower blood glucose even in the face of insulin resistance.

Newer drug classes include: (1) thiazolidinediones, which increase tissue insulin sensitivity by affecting gene expression; (2) α-glucosidase inhibitors, which interfere with absorption of some glucose containing nutrients, thereby reducing (or at least slowing) the amount of glucose absorbed; (3) meglitinides, which stimulate insulin release (nateglinide, repaglinide, and their analogs) quickly—they can be taken with food, unlike the sulfonylureas which must be taken prior to food (sometimes some hours before, depending on the drug); and (4) peptide analogs, which work in a variety of ways:
Incretin mimetics which increase insulin output from the beta cells among other effects. These includes the Glucagon-like peptide (GLP) analog exenatide, sometimes referred to as lizard spit as it was first identified in Gila Monster saliva;
Dipeptidyl peptidase-4 (DPP-4) inhibitors increase Incretin levels (sitagliptin) by decreasing their deactivation rates;
Amylin agonist analog, which slows gastric emptying and suppresses glucagon (pramlintide).

### Oral Drugs

A systematic review of randomized controlled trials found that metformin and second-generation sulfonylureas are the preferred choices for most with Type 2 diabetics, especially those early in the course of the disease. Failure of response after a time is not unknown with most of these agents: the initial choice of anti-diabetic drug has been compared in a randomized controlled trial which found “cumulative incidence of monotherapy failure at 5 years to be 15% with rosiglitazone, 21% with metformin, and 34% with glyburide”. Of these, rosiglitazone users showed more weight gain and edema than did non-users. Rosiglitazone may increase risk of death from cardiovascular causes though the causal connection is unclear. Pioglitazone and rosiglitazone may also increase the risk of fractures. For patients who also have heart failure, metformin may be the best tolerated drug.

The variety of available agents can be confusing and the choice of drugs for Type 2 diabetics is rarely straightforward and in most instances has elements of repeated trial and adjustment. Moreover, the side effects of many of these drugs (e.g., nausea, upset stomach, fluid retention and hypoglycemia) can frustrate and undermine the strict regiments required for treatment.

### Insulin Injections

If antidiabetic drugs fail (ie, the clinical benefit stops), insulin therapy may be necessary—usually in addition to oral medication therapy—to maintain normal or near normal glucose levels. Typical total daily dosage of insulin is 0.6 U/kg. But, of course, best timing and indeed total amounts depend on diet (composition, amount, and timing) as well the degree of insulin resistance.

### Surgical Intervention

Another treatment option considered for diabetics, particularly those that also suffer from obesity, is surgery. Gastric bypass procedures are currently considered an elective procedure with no universally accepted algorithm to decide who should have the surgery. In the diabetic patient, certain types result in 99-100% prevention of insulin resistance and 80-90% clinical resolution or remission of Type II diabetes. In 1991, the NIH (National Institute of Health) Consensus Development Conference on Gastrointestinal Surgery for Obesity proposed that the body mass index (BMI) threshold to consider surgery should drop from 40 to 35 in the appropriate patient. More recently, the American Society for Bariatric Surgery (ASBS) and the ASBS Foundation suggested that the BMI threshold be lowered to 30 in the presence of severe co-morbidities. More debate has flourished about the role of gastric bypass surgery in Type 2 diabetics since the publication of The Swedish Obese Subjects Study. The largest prospective series showed a large decrease in the occurrence of Type II diabetes in the post-gastric bypass patient at both 2 years at 10 years.

A study of 20-years of Greenville gastric bypass patients found that 80% of those with Type 2 diabetes before surgery no longer required insulin or oral agents to maintain normal glucose levels. Weight loss occurred rapidly in many people in the study who had had the surgery. The 20% who did not respond to bypass surgery were, typically, those who were older and had had diabetes for over 20 years.

In January 2008, The Journal of the American Medical Association (JAMA) published the first randomized controlled trial comparing the efficacy of laparoscopic adjustable gastric banding against conventional medical therapy in the obese patient with type 2 diabetes. Laparoscopic Adjustable Gastric Banding results in remission of Type 2 diabetes among affected patients diagnosed within the previous two years according to a randomized controlled trial.

Despite the advances of modern medicine, there are still many side effects and risks with currently available pharmaceutical and surgical treatments and the clinically preferred approach for treating type 2 diabetes and managing blood glucose is still self management through education, diet and exercise. As such, there continues to be a need for products and technologies that can help diabetics manage their blood sugar and achieve balance in their lives.

### DETAILED DESCRIPTION

The following detailed description is merely exemplary in nature and is not intended to limit the invention or the application and uses of the invention. Furthermore, there is no intention to be bound by any theory presented in the preceding background section or following detailed description section.

### Overview

A food additive, soluble powder, beverage and/or pharmaceutical agent for stabilizing and balancing blood glucose in hyperglycemic, diabetic and/or pre-diabetic patients is provided. This formulation may be used as a dietary supplement to supplant the need for or reduce the daily requirement of drugs for managing chronic disease conditions or disorders such as hypercholesterolemia, hyperlipidemia, hypertension, diabetes and prediabetic syndrome.

The composition of the present application comprises a digestible formulation of inulin, which includes an inulin, a sucrose and an amylase enzyme. Additional formulation constituents optionally include potassium, dimethyl sulfoxide, lentinan and D-eritadenine.

### Inulin

Inulins comprise a group of naturally occurring polysaccharides (several simple sugars linked together) produced by many types of plants. Inulins belong to a class of fibers known as fructans. Inulin is used by some plants as a means of storing energy and is typically found in the roots or rhizomes of these plants. Sources of inulin include, dandelion (taraxacum officinale), wild yam (dioscorea spp.), artichoke (helianthus tuberosus), chicory, (cichorium intybus), jicama (pachyrhizus erosus), burdock (articium lappa), onion (allium cepa), garlic (allium sativum), agave (agave spp.).

Inulin acts as a soluble fiber providing the type of bulk that aids the body's absorption of calcium and magnesium in the small intestine. In the large intestine, inulin is broken down by beneficial bacterial through fermentation to yield short chain fatty acids, including many different peptides. These beneficial products are believed to aid in the prevention of colorectal cancer among other conditions, disorders and diseases.

Inulin is conventionally referred to as a prebiotic, a non-digestible food item that stimulates the growth of beneficial bacteria existing naturally in the colon. These helpful bacteria are referred to as probiotics. Probiotic bacteria, specifically bifidobacteria and lactobacilli, promote health by inhibiting the growth of harmful pathogens thus reducing the potential for infections. Other potential benefits of prebiotics may include the reduction of the risk of colorectal cancer. Additionally, inulin has been linked to improving the quality of sleep.

The specific therapeutic impact of inulin largely depends on the molecular weight and chain length of inulin molecules ingested by the body. The size of the inulin chain helps determine which part of the gut and intestine will receive the dietary fiber. Since specific parts of the intestine produce certain peptides, the size of the inulin chain can affect the type of peptide produced. For example, short chains of inulin, which are most extensively fermented in the caecum and in the proximal colon, generally increase the levels of mRNA proglucagon and glucagon-like peptide 1 (GLP-1).

As shown in the below figure, the fermentation of oligofructose (OFS), an inulin-type fructane, stimulates the production of proglucagon and GLP-1 to yield numerous therapeutic effects. The increase in GLP-1 levels decreases glycemia and increases pancreatic insulin, thereby improving glucose tolerance. The peptides produced also modulate the vagal nerve to suppress appetite. The development of fat mass is also reduced. Thus, the consumption, ingestion and fermentation of a short-chain inulin can provide numerous therapeutic benefits, particularly to those who are obese, hyperglycemic, diabetic and/or pre-diabetic.

### Sucrose

Sucrose (common name: table sugar, also called saccharose) is a disaccharide (glucose+fructose) with the molecular formula C₁₂H₂₂O₁₁. Its systematic name is α-D-glucopyranosyl-(1→2)-β-D-fructofuranose. It is best known for its role in human nutrition and is formed by plants but not by other organisms. Glucose (Glc), a monosaccharide (or simple sugar), is an important carbohydrate in biology. Fructose (or levulose) is a simple sugar (monosaccharide) found in many foods and is one of the three most important blood sugars along with glucose and galactose. Sucrose is the most important sugar in plants, and can be found in the phloem sap. It is generally extracted from sugar cane or sugar beet and then purified and crystallized. Other (minor) commercial sources are sweet sorghum and sugar maples.

Since ingesting and metabolizing sucrose can quickly raise blood glucose, it would be generally be considered counterintuitive to include sucrose in a preparation for stabilizing blood sugar. However, since there is a possibility that the inulin could cause a significant drop in blood glucose, a relatively small amount of sucrose can be useful to mitigate this risk. The sucrose can be derived from any source, including vegetables such as carrots, corn, potatoes, beets, sugar peas, etc. or fruits such as peach, apple, mango, berries, etc. Alternatively, honey can also be used.

### Amylase

Amylase is an enzyme that breaks starch down into sugar. Amylase is present in human saliva, where it begins the chemical process of digestion. Foods that contain much starch but little sugar, such as rice and potato, taste slightly sweet as they are chewed because amylase turns some of their starch into sugar in the mouth. The pancreas also makes amylase (alpha amylase) to break down dietary starch into di- and trisaccharides which are converted by other enzymes to glucose to supply the body with energy. Plants and some bacteria also produce amylase. All amylases are glycoside hydrolases and act on α-1,4-glycosidic bonds.

One form of amylase, β-amylase (e.g., maltohydrolase, glycogenase, saccharogen amylase, etc.) is also synthesized by bacteria, fungi, and plants. Working from the non-reducing end, β-amylase catalyzes the hydrolysis of the second α-1,4 glycosidic bond, cleaving off two glucose units (maltose) at a time. During the ripening of fruit, β-amylase breaks starch into sugar, resulting in the sweet flavor of ripe fruit. Both are present in seeds; β-amylase is present prior to germination, whereas α-amylase and proteases appear once germination has begun. Cereal grain amylase is key to the production of malt. Many microbes also produce amylase to degrade extracellular starches. Animal tissues do not contain β-amylase, although it may be present in microorganisms contained within the digestive tract.

Amylase is useful in the present preparation to break down long chain inulin molecules into shorter chains. More specifically, daikon radish, which is rich in β-amylase, can be added to the preparation to create shorter inulin chains that will illicit the production of GLP-1 when fermented in the intestine.

### Potassium

Potassium is a chemical element. It has the symbol K (Latin: kalium, from Arabic: qalīy), atomic number 19, and atomic mass 39.0983. A diet that contains sufficient amounts of potassium may reduce the risk of high blood pressure and stroke. A decrease in muscular strength is often due to a lack of potassium in the diet. Diets sufficient in potassium helps soothe feelings of anxiety, irritability and stress and also protect against heart disease, hypoglycemia, diabetes, obesity and kidney disease. Potassium also helps keep muscles strong and bowels regular as well as protect against the blood pressure-boosting properties of sodium. Some foods that are high in potassium content include potatoes, beet greens, white beans, prunes, bananas, tomatoes, peaches, radishes and apricots.

### Dimethyl Sulfoxide

Dimethyl sulfoxide (aka DMSO, methyl sulfoxide, methylsulfinylmethane) is a chemical compound with the formula (CH₃)₂SO. DMSO, is a colorless, sulfur-containing organic liquid compound with a faint scent of sulfur and mixes readily with a wide range of water-insoluble and water-soluble substances. DMSO is rapidly absorbed into the body if ingested and if simply touched by the hands very quickly produces a garlic-like taste. DMSO occurs naturally in many plants including vegetables, grains, fruits, and even in some animal products, but is commercially produced as a by-product of wood pulping.

In the medical field DMSO is predominantly used as a topical analgesic, a vehicle for topical application of pharmaceuticals, as an anti-inflammatory and an antioxidant. Because DMSO increases the rate of absorption of some compounds through organic tissues including skin, it can be used as a drug delivery system. It is frequently compounded with antifungal medications, enabling them to penetrate not just skin but also toe and fingernails.

In the past DMSO has been sold in vitamin stores and used both externally and internally, marketed as treatment for aches and pains. Although DMSO has been examined for the treatment of numerous conditions and ailments, it is only approved by the FDA for the palliative treatment of interstitial cystitis. DMSO is also known to have a diuretic effect in helping the body to increase waste removal via urine excretion.

### Lentinan

Lentinan (C₄₂H₇₂O₃₆) is a complex polysaccharide that possesses immuno-stimulating antitumor properties. Lentinan is found in very low concentrations in fresh shiitake mushrooms (Lentinula edodes). In 1 study, 200 kg of fresh mushrooms yielded 31 g of lentinan (0.02%). Lentinan is a water-soluble, beta-1,3 glucan polysaccharide characterized by beta-1,6 branched glucan linkages. At least 5 additional polysaccharides have been isolated from L. edodes. Lentinan is a high molecular weight polysaccharide in a triple helix structure, containing only glucose molecules with mostly (1-3)-β-D-Glucan linkages in the regularly branched main chain with two β (1,6)-D-glucopyranoside branchings for every five β-(1,3)-glucopyranoside linear linkages.

The antitumor activity of lentinan has been recognized for almost 30 years. Because a number of naturally occurring polysaccharides had previously been found to have antitumor activity, lentinan was considered for detailed evaluation. In addition to antitumor activity, lentinan also possesses immune-regulatory effects, antiviral activity, antimicrobial properties and cholesterol-lowering effects.

### Eritadenine

Eritadenine is a compound found in shiitake mushrooms. It is generally considered useful in lowering blood cholesterol.

### DESCRIPTION OF REPRESENTATIVE EMBODIMENTS

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention, which is defined by the claims.

In an exemplary embodiment of the present invention, the formulation comprises inulin, sucrose and amylase. It is desirable that the inulin component be a short chain inulin. For example, it is desirable that the inulin chain be less than about DP 25. Additionally or alternatively, it is desirable that the inulin chain be less or equal to about DP 12. The formulation may also comprise potassium, dimethyl sulfoxide, lentinan and eritadenine.

Although these constituents can be synthesized in a laboratory, it is desirable that they be derived from naturally occurring plants and vegetables. For example, one embodiment may comprise burdock root, carrots, radish, radish greens and shiitake mushrooms. Each vegetable provides at least one constituent of the formulation. Burdock root provides inulin and potassium; carrots supply sucrose and β-carotene; daikon radish and radish greens provide potassium and β-amylase; and shiitake mushrooms provide lentinan and eritadenine. These vegetables can be used in extract or powdered form, but it is generally preferred that fresh vegetables be used.

The present formulation of inulin, sucrose and β-amylase may also be obtained from various alternate combinations of the aforementioned vegetable sources. For example, inulin sources, including chicory, dahlia, asparagus, and Jerusalem artichokes, can be combined with one or more sources of sucrose, including sugar peas, beets, corn, potatoes, and other simple starches, and one or more sources of β-amylase, including green vegetables such as spinach, lettuce, beet greens, and mustard greens.

The above mentioned vegetables can be combined in many different quantities or relative concentrations. However, it is generally preferred that the burdock root and carrots be added in approximately equal amounts by mass (i.e., 1 to 1 mass ratio) to maintain a balance between inulin and sucrose. It is also generally preferred that the radish be added in quantities approximately twice that of the burdock root (i.e., 2 to 1 mass ratio) so that sufficient amylase is present to break the inulin into shorter chains.

The formulation can be prepared using a unique production process. The vegetables are first prepared for cooking by being cleaned. The vegetables are then added with water to a cooking unit. The cooking unit may comprise any type of cooking device, including a boiler, pressure cooker or steam closet. The vegetables can be added whole or first chopped, sliced or grated before being added to the cooking unit. The vegetables and water are cooked in the cooking unit for approximately 10 to 11 hours at about 120 to 130 degrees Fahrenheit. Cooking the vegetables slowly allows the inulin to leach out of the burdock root and the β-amylase from the radish to break the inulin down into shorter chains. The relatively low temperature provides an optimal environment for the enzymatic action of the amylase to effectively break down the inulin. At the conclusion of the cooking period, the temperature is raised to stop the enzymatic action. It is desirable that the temperature be raised to about at least 185 degrees Fahrenheit.

At the conclusion of the cooking sequence, the contents of the cooking unit are pressed to extract the juices from the vegetables. This juice can be readily packaged for distribution via bottling or canning. Alternatively and desirably, the juice is reduced to a more concentrated form. The concentration allows for more cost effective transportation of the juice. Although any concentration may be useful, it is preferred that the juice be concentrated from about 3% soluble solids to about 30% soluble solids. More specifically, it is generally desirable that the juice be concentration to about 20% soluble solids.

The juice can be concentrated any number of ways. It can be accomplished using a rota-vaporator. Alternatively, reverse osmosis can be used to concentrate the juice. Still alternatively, a spin dryer can be used for the concentration step. Once concentrated, the juice is frozen in secure containers for transportation.

As a concentrate, the formulation can be commercialized into several products. For example, the concentrate can be sold as an extract to be consumed directly or added as a supplement to other foods (e.g., soup, stew, etc.) or beverages (e.g., coffee, tea, water, soda, etc.). The concentrate can also be sold in frozen form as a popsicle.

Alternatively and desirably, the concentrate is further reduced to a powder. Powderization can occur via several methods. Spray drying is a common and popular method for transforming liquid into powder by drying a liquid through a hot gas. This process is generally a one-step, rapid process that eliminates additional processing. The liquid concentrate is pumped through an atomizer device that produces fine droplets into the main drying chamber. The droplets allow a much larger surface area of the liquid to be exposed to the hot air stream, thereby facilitating a quick dehydration. Several types of atomizers can be used, including rotary/centrifugal (spinning disks), single fluid nozzles, two-fluid nozzles and ultrasonic nozzles. Other (non-spray) methods for powderization include batch drying, drum drying, lyophilization (freeze drying) and solar drying.

To facilitate drying in some or all of the above dehydration methods, it may be useful to add a drying additive such as Gum Arabic powder to the concentrate solution prior to powderization. It is desirable that the Gum Arabic powder be added to the concentrate solution in equal amounts (i.e., 1 to 1 ratio). Alternatively or additionally, other additives may be used to facilitate drying, including cellulose, carboxymethyl cellulose, mannitol, maltodextrin and dextrin.

To maintain the stability and shelf life of the powder, it may be desirable to add preservatives during the preparation process. These preservatives include calcium propionate, sodium nitrate, sodium nitrite, sulfites (e.g., sulfur dioxide, sodium bisulfite, potassium hydrogen sulfite, etc.), an EDTA (e.g, disodium EDTA) and ascorbic acid.

Once in a powder form, the formulation can be packaged and consumed in a number of commercial forms. For example, the powder can be consumed directly or provided as a food additive. Alternatively, the power can be compacted into a pill or tablet or encapsulated into capsules or microcapsules to be taken as a daily supplement.

Alternatively and preferably, the powder can be used as a solute to be added to a beverage (e.g., water, coffee, tea, soda, juice, etc.). The formulation in powder form can even be used as a powdered drink mix to be added to water. For example, natural and/or artificial flavoring can be added to the powdered formulation such that the resulting mix provides a tasty beverage when mixed with water or some other solvent. The powdered drink mix can be provided in a large container or in daily use packets. The drink mix could also be roll of soluble tablets with each tablet providing a single serving of the formulation.

To increase the sweet taste of the formulation and/or improve its solubility, erythritol can be added to the formulation. Erythritol is a polyol (sugar alcohol) which is commonly used as a sweetener in low carbohydrate foods. Although it tastes sweet, erythritol cannot be metabolized by the body and, accordingly, does not have a meaningful impact on blood glucose when consumed. Erythritol in sufficient quantities can also enhance the solubility of dehydrated compounds. For example, adding erythritol to the present formulation following spray dehydration. Although any quantity of erythritol may increase solubility of the formulation, it is generally desirable to add about 3 units of erythritol to every 2 units of formulation (i.e., 1.5:1 mass ratio). This ratio will help avoid clumping when the dried formulation is added to solvent, such as water.

To achieve the optimal therapeutic benefit of the present invention, it is generally preferred, but not required, that the formulation be consumed on a daily basis. For example, taking approximately 4 grams of the formulation each day may improve blood glucose control in many individuals, including diabetic and pre-diabetic patients. Taking the formulation at nighttime will also allow the consumer to take advantage of the sleep benefits associated with inulin.

In one clinical study, 30 patients were provided a two week supply of formulation in beverage form and were asked to consume 8 ounces of the beverage each night for two weeks and record fasting blood glucose readings based on self-administered glucose meter test. 80% of participants responded favorably with steady reductions in blood glucose. In responders, the average reduction in blood glucose was approximately 40 points.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. When the claims use the word “or” in reference to a list of two or more items, that word covers all of the following interpretations of the word: any of the items in the list, all of the items in the list, and any combination of the items in the list.

The above detailed descriptions of embodiments of the invention are not intended to be exhaustive or to limit the invention to the precise form disclosed above. Although specific embodiments of, and examples for, the invention are described above for illustrative purposes, various equivalent modifications are possible within the scope of the invention, as those skilled in the relevant art will recognize. For example, while steps are presented in a given order, alternative embodiments may perform steps in a different order. The various embodiments described herein can also be combined to provide further embodiments.

In general, the terms used in the following claims should not be construed to limit the invention to the specific embodiments disclosed in the specification, unless the above detailed description explicitly defines such terms. While certain aspects of the invention are presented below in certain claim forms, the inventors contemplate the various aspects of the invention in any number of claim forms. Accordingly, the inventors reserve the right to add additional claims after filing the application to pursue such additional claim forms for other aspects of the invention.

I/We claim:

1. A therapeutic digestible formulation for consumption by a patient comprising:
an inulin;
a sucrose; and
an amylase;
wherein the inulin is configured to lower blood glucose in the patient and wherein the sucrose is present in sufficient amount to at least partially counteract the blood glucose lowering effect of the inulin.

2. The formulation of claim 1 wherein the inulin is comprised of a short chain inulin.

3. The formulation of claim 2 wherein the short chain inulin comprises a chain length less than about DP 25.

4. The formulation of claim 2 wherein the short chain inulin comprises a chain length less than about DP 12.

5. The formulation of claim 1 wherein the amylase comprises a β-amylase.

6. The formulation of claim 1, further comprising at least one of potassium, dimethyl sulfoxide, lentinan and D-eritadenine.

7. The formulation of claim 1, further comprising potassium, dimethyl sulfoxide, lentinan and D-eritadenine.

8. The formulation of claim 1 wherein the inulin is derived from at least one of dandelion, wild yam, artichoke, chicory, jicama, burdock, onion, garlic, and agave.

9. The formulation of claim 1 wherein the inulin, sucrose, and amylase are obtained from vegetables.

10. The formulation of claim 1 wherein the inulin is obtained from burdock root, the sucrose is obtained from carrots and the amylase is obtained from daikon radish.

11. A method of preparing a therapeutic formulation for oral consumption, the method comprising:
combining a vegetable mixture comprising burdock root, carrots and daikon radish;
cooking the vegetable mixture for a predetermined period of time at a predetermined temperature; and
pressing the vegetable mixture to extract a vegetable juice.

12. The method of claim 11 wherein cooking the vegetable mixture for a predetermined period of time at a predetermined temperature comprises cooking the vegetable mixture for about 10 to about 11 hours at a temperature of about 120 to about 130 degrees Fahrenheit.

13. The method of claim 12, further comprising raising the temperature of the vegetable mixture to at least about 185 degrees Fahrenheit before pressing the vegetable mixture.

14. The method of claim 11 wherein combining burdock root and carrot further comprises combining approximately equal amounts by mass of burdock root and carrot.

15. The method of claim 11 wherein combining daikon radish and burdock root further comprises combining approximately twice as much daikon radish as burdock root by mass.

16. The method of claim 11, further comprising concentrating the vegetable juice to a concentrate of about 3% soluble solids to about 30% soluble solids.

17. The method of claim 16 wherein concentrating the vegetable juice further comprises concentrating the vegetable juice to a concentrate of about 20% soluble solids.

18. The method of claim 16, further comprising drying the concentrate to a powdered mixture.

19. The method of claim 18 wherein drying the concentrate to a powdered mixture comprises introducing a drying additive to the concentrate.

20. The method of claim 18, further comprising adding erythritol to the powdered mixture.
